(19) United States
(12) Patent Application Publication
(10) Pub. No.: US 2009/0137844 A1
(43) Pub. Date: May 28, 2009

(54) Crystallization process

(75) Inventors: Arne Grumann, Kauniainen (FI); Soini Huhta, Espoo (FI); Eila Luukkonen, Espoo (FI); Tuomas Koiranen, Helsinki (FI)

Correspondence Address: BIRCH STEWART KOLASCH & BIRCH, PO BOX 747, FALLS CHURCH, VA, 22040-0747, US

(73) Assignee: Fermion Oy, Espoo (FI)

(21) Appl. No.: 11/921,245

(22) PCT Filed: Jun. 14, 2006
(86) PCT No.: PCT/FI2006/000201

§ 371 Date: Jan. 17, 2008

Related U.S. Application Data
(60) Provisional application No. 60/690,515, filed on Jun. 15, 2005.

(51) Int. Cl. C07C 311/16

(52) U.S. Cl. 564/86

(57) ABSTRACT

The invention relates to a process for the production of tamsulosin hydrochloride crystals where the crystal size distribution is controlled by controlling the temperature where hydrochloric acid is added to the reaction mixture.

The compound 5-[2-[[2-(2-ethoxyphenoxy)ethyl]amino]propyl]-2-methoxybenzene-sulfonamide, which has the INN name tamsulosin and the chemical formula (I)

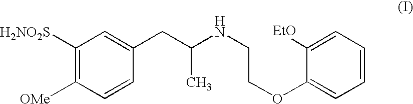

is a commercially marketed pharmaceutically active substance useful for treatment of cardiac insufficiencies and benign prostatic hyperplasia. It was first disclosed in U.S. Pat. No. 4,731,478. The commercially marketed product is the hydrochloride salt of the (R)-(−)-enantiomer of tamsulosin.

The preparation of tamsulosin as crystalline hydrochloride is described e.g. in WO 2004/06582, WO 2004/022532, WO 03/037851, WO 03/0377850, WO 03/035608, EP 34432 and CA 1282077. Solvents used in crystallization have been e.g. ethanol or methanol. Crystal sizes have not been mentioned.

Tamsulosin HCl is administered as sustained release capsules containing only 0.4 mg of the active ingredient. It is important that the crystals of tamsulosin HCl are small so that the active ingredient is evenly spread among additional ingredients. It is possible to make a product with a suitable particle size distribution by micronization, but the losses in this process may be considerable. Therefore it is advantageous if crystals of suitable particle size are obtained directly from the process. The inventors have surprisingly noticed that it is possible to control the particle size distribution by using suitable solvents and adding the salt forming reagent in a suitable temperature.

### SUMMARY OF THE INVENTION

The present invention relates to a process for producing tamsulosin hydrochloride crystals by a process which produces desired, in advance defined, particle size distribution. This process comprises slurrying tamsulosin base in a mixture of an alcoholic solvent and water, adding diluted hydrochloric acid, and isolating tamsulosin hydrochloride formed. The crystal size distribution can be regulated by using different temperatures during the addition of hydrochloride to the slurry or solution of tamsulosin base.

Another object of the present invention is tamsulosin hydrochloride where more than 90% of the crystals are below 60 micrometers analyzed by laser diffraction.

### DETAILED DESCRIPTION OF THE INVENTION

Particle size distribution of tamsulosin HCl has not been controlled or even mentioned in any of the publications mentioned above. The present invention is based on the discovery that if tamsulosin is crystallized from alcoholic solvents, the crystals are the smaller the lower the temperature is during the addition of hydrochloric acid.

Tamsulosin base used as a starting material may be made e.g. as described in U.S. Pat. No. 4,731,478 or by any other method know in the art.

The typical crystallization process includes the steps of heating the solution or slurry of tamsulosin base in a crystallization solvent mixture to a reflux temperature, cooling to the desired addition temperature, adding the salt forming reagent, hydrogen chloride, mixing the slurry and isolating tamsulosin hydrochloride and drying it.

Addition temperature is the temperature where the slurry is kept during the addition of hydrochloric acid.

Addition temperatures of the invention vary between 0° C. and 80° C. due to the boiling point of the solvent used and the crystal size distribution desired.

The alcoholic solvent used in the crystallization process is preferably isopropanol, but also other lower alcohols, e.g. methanol or ethanol can be used. Water is added to the solvent in order to increase the solubility, while tamsulosin is poorly soluble in alcoholic solvents. The ratio of water to alcohol used is about 1:45 to 1:30. Tamsulosin base and the hydrochloride produced can be either racemic or enantiomerically enriched.

Even smaller amounts of water may be used, and then the ration of water to alcohol becomes more decisive factor to the crystal size distribution. This is especially important for larger crystal sizes, and accordingly in using higher temperatures during the addition of hydrochloric acid.

Crystallization or cooling times do not have any significant influence on the crystal size distribution. The mixture may be warmed after the addition of hydrochloric acid or the process may be continued at the addition temperature. The mixture is stirred at the selected temperature from 10 minutes to several hours, usually 15 to 30 minutes is enough. Thereafter the mixture will be cooled, optionally mixed some time, and the crystals are isolated from the mixture by any suitable method known in the art, e.g. by filtration.

Particle size distribution is analyzed visually using light microscope or by laser diffraction using the following method:

Saturated 2-Propanol: To 250 ml of 2-propanol is added approx 1 g of tamsulosin HCl powder and sonicated at room temperature about 30 min. The mixture is let stand overnight, hereafter it is filtered through 0.22 μM membrane filter.
Sample Preparation About 30 mg of tamsulosin HCl is weighed in a 25 ml decanter. 2-4 drops of saturated 2-propanol is added to the sample and stirred with a glass rod. After that 4 ml of saturated 2-propanol is added and the sample is kept in ultra sound for 30 seconds before measuring volumetric particle size distribution using Coulter LS230, Laser Diffraction Particle Size Analyzer.

As long as the temperature of the mixture is below about 70° C., the tamsulosin base is mainly in solid form and there is no solution, but a thick slurry. Hydrochloric acid is added to this slurry in the predefined addition temperature in which the crystals of the desired size are produced. If e.g. the temperature where the hydrochloride is added is below about 30° C., 90% of the crystals are below 15 micrometers, whereas if the temperature is higher, e.g. 80° C., 90% of the crystals will be below 250 micrometers (light microscope). Analyzed by laser diffraction, if the addition temperature is about 20° C., 90% of the crystals are below about 20 micrometers, and if the temperature is about 40° C., 90% of the crystals are below about 60 micrometers.

### EXAMPLES

### Example 1

Tamsulosin base (168 g), 2-propanol (1500 ml) and water (50 ml) were charged into a reaction vessel. The mixture was warmed to reflux and the obtained solution was filtered to remove inorganic material. The filtrate was cooled to 40° C. (Tamsulosin base precipitated during cooling). To the suspension was added 12% hydrochloric acid (125 ml) during 2-5 minutes. The mixture was warmed to 55° C. and stirred for 20 minutes at 55° C. Then the mixture was cooled to 0° C. and stirred for 1 hour. The crystals were filtered, washed with 2-propanol (200 ml) and dried. Yield 174 g (95%).

In table 1 below there are the results of experiments using different addition temperatures. Procedure is the same as above.

TABLE
Dependence of the crystal particle size on addition temperature of
HCl, particle size determination by microscope (visually observed)
or laser diffraction (volumetric particle size distribution).

| | | Size of particle | Particle size distribution |
|---|---|---|---|
| HCl added at | | (light microscope) | (laser diffraction) |
| 0° | C. | 90% < 3 μm | LDP 90 = 12 μm |
| | | | LDP 50 = 3 μm |
| | | | LDP 10 = 1 μm |
| 10° | C. | 90% < 5 μm | LDP 90 = 10 μm |
| | | | LDP 50 = 5 μm |
| | | | LDP 10 = 1 μm |
| 20° | C. | 90% < 10 μm | LDP 90 = 17 μm |
| | | | LDP 50 = 7 μm |
| | | | LDP 10 = 2 μm |
| 30° | C. | 90% < 15 μm | LDP 90 = 29 μm |
| | | | LDP 50 = 15 μm |
| | | | LDP 10 = 4 μm |
| 40° | C. | 90% < 30 μm | LDP 90 = 55 μm |
| | | | LDP 50 = 29 μm |
| | | | LDP 10 = 9 μm |
| 80° | C. | 90% < 250 μm | No analysis available |

### Example 2

Tamsulosin base (5 g), 2-propanol (43.5 ml) and water (1.5 ml) were charged into a reaction vessel. The mixture was warmed to reflux so that crystals were dissolved. The solution was cooled to 40° C. (tamsulosin base precipitated during cooling). To the suspension was added 15% hydrochloric acid (3.7 ml) during 1-5 minutes. The mixture was warmed to 55° C. and stirred for 20 minutes at 55° C. Then the mixture was cooled to 20° C. and stirred at least for 2 hours. The crystals were filtered, washed with 2-propanol (10 ml) and dried. Yield 5.18 g (95%). The crystal size distribution was analyzed by laser diffraction:

LDP 90=36 μm

LDP 50=20 μm

LDP 10=5 μm

### Example 3

The experiment was repeated using 20° C. as a HCl addition temperature. The crystal size distribution analyzed by laser diffraction was the following:

LDP 90=29 μm

LDP 50=14 μm

LDP 10=3 μm

1. Process for the preparation of tamsulosin hydrochloride comprising:
a) slurrying tamsulosin base in a mixture of an alcoholic solvent and water,
b) adding diluted hydrochloric acid, and
c) isolating tamsulosin hydrochloride formed.

2. Process for the preparation of tamsulosin hydrochloride comprising:
a) slurrying tamsulosin base in a mixture of an alcoholic solvent and water,
b) adding diluted hydrochloric acid in a predefined temperature, and
c) isolating tamsulosin hydrochloride formed.

3. A process of claim 1 or 2 wherein the alcoholic solvent used is isopropanol.

4. A process of claim 2 where the predefined temperature is between 0° C. and 80° C.

5. A process according to claim 4 where the predefined temperature is between 2O° C. and 60° C.

6. Tamsulosin hydrochloride with crystal size distribution where more than 90% of the crystals are below 60 micrometers analyzed by laser diffraction.

7. Tamsulosin hydrochloride crystals made according to claim 1 or 2 where more than 90% of the crystals are below 60 micrometers analyzed by laser diffraction.

8. A process according to claim 2 where the predefined temperature is about 4O° C.

9. Tamsulosin hydrochloride produced according to claim 8, wherein more than 90% of crystals are smaller than 60 micrometers analyzed by laser diffraction.
